(19) United States
(12) Patent Application Publication
(10) Pub. No.: US 2003/0187381 A1
(43) Pub. Date: Oct. 2, 2003

(54) Bioresorbable foam packing device and use thereof

(75) Inventors: Keith E. Greenawalt, Milton, MA (US); Dana A. Oliver, Jacksonville, FL (US)

Correspondence Address: Kent H. Cheng, Esq., Cohen, Pontani, Lieberman & Pavane, 551 Fifth Avenue, Suite 1210, New York, NY, 10176, US

(73) Assignee: Genzyme Corporation

(21) Appl. No.: 10/330,787

(22) Filed: Dec. 27, 2002

Related U.S. Application Data
(60) Provisional application No. 60/343,949, filed on Dec. 28, 2001.

(51) Int. Cl. A61F 13/20

(52) U.S. Cl. 604/11

(57) ABSTRACT

This invention provides a bioresorbable foam packing device for post-operative use, especially to separate and prevent adhesions between mucosal surfaces in the nasal cavity, to help control minimal bleeding, and to prevent lateralization of the middle turbinate.

### RELATED APPLICATIONS

This application claims priority from U.S. Provisional Patent Application Serial No. 60/343,949 which was filed on Dec. 28, 2001.

### BACKGROUND OF THE INVENTION

1. Field of the Invention

This invention relates to surgery techniques, especially surgical procedures of the nasal and sinus cavity. In particular, it relates to a bioresorbable foam packing for post-operative use to separate tissue surfaces and prevent adhesions, especially between mucosal surfaces in the nasal cavity, to help control minimal bleeding, and to prevent lateralization of the middle turbinate.

2. Description of the Related Art

Recent developments in the field of surgical techniques and medical devices have provided the skilled otorhinolaryngologist with instrumentation and methods to perform complex paranasal sinus surgical procedures. Improved visualization of the nasal cavity and paranasal sinuses now makes these anatomical areas more accessible to the endoscopic surgeon. Surgical guidelines for performing these operations are described in “Endoscopic Paranasal Sinus Surgery” by D. Rice and S. Schaefer, Raven Press, 1988) and in the writings of M. E. Wigand, Messerklinger and Stamberger. Various procedures, such as anterior and posterior ethmoidectomy, sphenoidectomy, maxillary antrostomy, frontal sinusotomy, etc., may be performed in these areas. Nasal and sinus surgeries are now common procedures with 500,000 to 700,000 performed in the United States every year.

Of these nasal and sinus surgeries, it is estimated that there is an 8% incidence of adhesion formation, with approximately 15% of these patients requiring revision surgery. A particular problem encountered by the endoscopic surgeon has been postoperative adhesion occurring between the middle turbinate and adjacent nasal areas, such as medial adhesion to the septum and lateral adhesion to the lateral nasal wall in the area of the ethmoid sinuses. Otherwise successful surgical procedures may have poor results in these cases. Some surgeons have proposed amputation of the lower half of the middle turbinate at the conclusion of surgery to avoid this complication, resulting in protracted morbidity (crust formation and nasal hygiene problems). The turbinate adhesion problem detracts from an otherwise refined endoscopic surgical procedure.

In an attempt to avoid adhesions, surgeons may often pack the operative site with nonfiber, hydratable and expandable packing, or other materials such as tampons. A “sinus pack” tampon, such as disclosed in U.S. Pat. No. 4,646,739, may be used for short term packing of the operative site; however, risk of “toxic shock syndrome” after only a day or two is significant. The use of post-operative packing, such as Merogel® nasal dressing and sinus stent, is reported to prevent lateralization of the middle turbinate while packing the osteomeatal complex. Merogel® comprises esters of hyaluronic acid, and is disclosed in U.S. Pat. No. 4,851,521. Packing can displace the middle turbinate in a medial direction and carries with it a significant risk of having the turbinate adhere to the nasal septum, with resultant airway obstruction. While various septal splints can prevent adhesions to the nasal septum, adhesions of the lateral aspect of the middle turbinate to the lateral ethmoid sinus wall are not prevented concurrently.

It is an object of the present invention to provide a process for making bioresorbable foam useful for preventing adhesion of tissues following surgery.

It is another object of the present invention to provide a process for making bioresorbable foam useful in nasal packing and stent devices.

It is another object of the present invention to provide a bioresorbable foam packing and stent device for application into the nasal cavity to prevent both nasal septum and side wall adhesion for at least seven days during healing.

It is another object to provide a bioresorbable foam packing device to help control bleeding following nasal or sinus surgery.

### SUMMARY OF THE INVENTION

Seprafilm® Bioresorbable Membrane (Genzyme Corporation, Cambridge, Mass.), a sodium hyaluronate/carboxymethylcellulose (“HA/CMC”) device, is approved for use in the United States for the reduction of the incidence and severity of post surgical abdominal and pelvic adhesion. Preparation of Seprafilm® and other HA/CMC materials are generally disclosed in U.S. Pat. Nos. 5,527,893; 5,017,229; and 4,937,270. Water insoluble gels can be made by combining hyaluronic acid, a polyanionic polysaccharide such as carboxymethylcellulose, and an activating agent under conditions sufficient to form a gel. However, there is no teaching in these patents or in the art at present as to the parameters of a specific, improved process of producing an HA/CMC foam having the proper physical characteristics that would allow its use for preventing tissue adhesion following surgery, and especially for its use as nasal packing and sinus stents. The novel HA/CMC foam, currently named Seprapack™ bioresorbable nasal packing and sinus stent, has been found useful in reducing adhesion formation following nasal and sinus surgery.

In particular, we have developed a novel, improved process of making a foam form of HA/polyanionic polysaccharide that exhibits the proper softness, flexibility, and degree of hydration and expansion necessary for use as a nasal packing material that is easily handled by the surgeon without breaking, can contour easily within the nasal cavity, can expand to at least 90% of its original dimensions upon hydration in order to hold open the nasal cavity, and has sufficient mass to prevent adhesion for approximately 3 to 5 days, and yet be significantly bioresorbed within 7 to 10 days. A “polyanionic polysaccharide” is a polysaccharide containing more than one negatively charged group, e.g., carboxyl groups at pH values above about pH 3.0. The polyanionic polysaccharide that is used to make the foam of the present invention includes, but is not limited to, carboxymethylcellulose, carboxymethylamylose, chondroitin-6-sulfate, chondroitin-4-sulfate, dermatin sulfate, alginate, heparin, and heparin sulfate. The preferred polyanionic polysaccharide is carboxymethylcellulose.

As used herein the term “HA” that is used to make the foam of the present invention means hyaluronic acid and any of its hyaluronate salts, including, for example, sodium hyaluronate (the sodium salt), potassium hyaluronate, magnesium hyaluronate, and calcium hyaluronate. HA may also be a chemical derivative of hyaluronic acid. When a HA/CMC composition of the present invention is made, the amount of HA and CMC may vary over a wide range, and is preferably 22 to 45% by volume of CMC and 49 to 73% by volume of HA. The HA/CMC composition may be made with or without an activating agent as disclosed in U.S. Pat. No. 5,527,893. A polyanionic polysaccharide is said to be “activated,” when it is treated in an aqueous mixture in a manner that renders the carboxyl groups on the polyanionic polysaccharide vulnerable to nucleophilic attack; and an “activating agent” is a substance that, in an aqueous mixture including a polyanionic polysaccharide, causes the polyanionic polysaccharide to become so activated. A useful activating agent is a carbodiimide, such as 1-ethyl-3-(3-dimethylaminopropyl) carbodiimide hydrochloride (“EDC”).

A novel nasal surgery method and medical device have been discovered, wherein the human middle turbinate, contiguous paranasal sinuses and/or nasal septum which have been subjected to surgical procedure(s) and/or trauma are protected by a flexible, bioresorbable foam packing.

The improved post-operative healing technique comprises applying to the post-operative middle turbinate a foam packing of sodium hyaluronate/carboxymethylcellulose. The foam packing functions to fill nasal/sinus cavities and to keep mucosal surfaces separate during the healing process. Shortly, after placement, the foam packing turns into a hydrated gel that is slowly resorbed into the body. During this time, the tamponade effect helps to control minimal bleeding normally associated with routine sinus surgery. The foam packing leaves the site of placement by natural elimination in approximately 7-10 days, or it may be aspirated from the cavity earlier at the discretion of the physician. Such natural elimination of the foam packing by bioresorption eliminates the need of a second surgical procedure to physically extract the packing from the nasal cavity, which risks reopening wounds.

The various features of novelty that characterize the invention are pointed out with particularity in the claims annexed to and forming a part of the disclosure. For a better understanding of the invention, its operating advantages, and specific objects attained by its use, reference should be made to the following descriptive matter in which there are illustrated and described preferred embodiments of the invention.

### DETAILED DESCRIPTION OF THE PRESENTLY PREFERRED EMBODIMENTS

Foams of the HA/CMC Seprapack™ material can be made by lyophilization (freeze drying). Lyophilization allows for a material to be frozen and then dried under high vacuum, during which the spaces occupied by ice crystals are replaced by voids or air pockets. This creates a highly porous solid structure with high void volume that is unattainable by conventional air-drying at elevated temperatures. These procedures are generally well known in the art. For example, Burns et al. U.S. Pat. No. 6,294,202 describes lyophilizing HA/CMC into thin sheets and combining with hydrophobic bioabsorbable polymers. Yannas et al., U.S. Pat. No. 4,280,954 and Dagalakis et al., 1980, J. Biomed. Mater. Res., V. 14, p. 511-528, describe methods of freeze drying collagen-polysaccharide composites and controlling pore structure. None of the prior teachings, however, provide the instant improved process for making foams of HA/CMC suitable in physical characteristics for use as a nasal packing or sinus stent device. As one aspect of the present invention, a novel, improved process has been developed to produce HA/CMC foams that are suitable for use as a nasal packing or sinus stent device. Examples of the novel, improved process for making HA/CMC foams is provided below.

1. First Example Of The Process

The starting material of the process, N-acylurea modified HA/CMC powder, was made as follows.

Sodium hyaluronate (0.4% w/w, 0.01M) and Aqualon-type CMC having a molecular weight of 250,000 and a degree of substitution in the range 0.65 to 0.90 (0.19% w/w, 0.01M) were mixed together in aqueous solution at room temperature. The pH of the mixture was adjusted to and maintained at pH 4.5-5.3 by addition of 1M HCl. To each 100 ml of this solution was added 0.67 g (0.04M) 1-ethyl-3-(3-dimethylaminopropyl) carbodiimide hydrochloride (“EDC”). During reaction with EDC, the pH of the solution was maintained at pH 4.7-4.8 by addition of 0.1M HCl and the reaction allowed to proceed for 1 hour, during which time a precipitate formed. The HA/CMC was further precipitated by the addition of ethanol. The precipitate was vacuum dried at or above room temperature to produce the HA/CMC powder.

The HA/CMC powder was resuspended in distilled water at a concentration of about 1.5 to about 3% weight/volume, preferably 2% weight/volume (e.g. 2g/100 ml) using a high shear mixer. The resuspended solution was metered into lyophilization trays with multiple cavities measuring approximately 4 cm×1.2 cm×1 cm and freeze-dried into solid foam plugs. Specifically, the shelf temperature of the lyophilizer was initially set at 0° C. and then thermally ramped to −4° C. at a rate of 0.4° C./min. and maintained at −4° C. for 60 min. The shelf temperature was then thermally ramped to −10° C. at a rate of 0.13° C./min. and maintained at −10° C. for 5 minutes. The shelf temperature was then ramped to −45° C. at a rate of 0.58° C./min., and held at −45° C. for 24.5 hours. The drying cycle was executed with a vacuum set point of 75 μm Hg with shelf temperature thermally raised to 0° C. at 0.75° C./min., and maintained for 5 hours. The shelf temperature was thermally raised to 40° C. at 0.22° C./min. and maintained for 8-32 hours. The resulting foam plugs were dehydrothermal treated (100° C. for 7 hours), exposed to air at a relative humidity of about 40% for at least about 4 hours, compressed (approximately 200-1000 psi for 1 to 2 minutes with a 0.3 cm spacer to prevent over-compression), packaged, and gamma-irradiated at 25-40 kGy.

Material prepared by this method can be used as a space-occupying stent to separate and prevent adhesions between mucosal surfaces in the nasal cavity, to help control minimal bleeding, and to prevent lateralization of the middle turbinate during the post-operative period following sinus surgery. Various processes for sterilizing the foam plugs may be used other than gamma-irradiation, including e-beam irradiation and ethylene oxide sterilization.

2. Second Example of the Process

A. Process Summary: Seprapack foam plugs were made from a 2.0% HA:CMC resuspension. This resuspension was metered into individual cells of a thermally stable tray and lyophilized to remove free water. The foam plug products were then dry heat treated and equilibrated at ambient temperature and relative humidity for at least 4 hours. The foam plugs were then compressed and packaged in hermetic pouches. After packaging, the foam plugs were terminally sterilized using gamma irradiation. The foam plugs were stored at ambient temperature in foil packaging.

B. Detailed Process Description

1. Resuspension

a. HA-CMC Powder Calculations

Determine the size of the metered resuspension, weigh out dry HA/CMC powder correcting for moisture. The HA/CMC powder is the same as that used as the starting material in the First Example.

b. Mixing

Weigh out the appropriate amount of water-for-injection required for resuspension. Using a low shear mixer or slow mixing rate at approximately 100 rpm, add the HA/CMC powder slowly to avoid clumping. Following complete introduction of the HA/CMC powder into the water-for-injection, high shear mix at approximately 10,000 rpm for a minimum of 10 minutes or until uniform to assure all clumps or agglomerates have been completely saturated into the water-for-injection.

c. Storage

Resuspension can be stored at 2-8° C. for 24 hours in a tightly sealed container.

2. Filling and Lyophilization

a. Filling

Lyophilization tray filling is preformed using a positive displacement peristaltic pump. Each lyophilization tray cell is filled with 6 mL of resuspension, which is approximately 1 cm in height. Light mixing is necessary to keep the resuspension homogenous. Utilizing a minimum size silicone tubing of 6 mm ID facilitates tray filling.

b. Lyophilization

Precool the Lyophilizer shelves in an Edwards 9 sq. ft. lyophilizer unit to approximately −50° C.±3° C. It is important to maintain a stable temperature.

Introduce the filled lyophilization trays into the lyophilizer and allow to freeze as quickly as possible. The foam plugs must be frozen rapidly in the lyophilizer to achieve the proper ice crystallization-this directly correlates to product flexibility. If the lyophilizer shelves are not cold enough or are cooling from room temperature and the freezing is slow, the product will be brittle and less desirable.

Maintain the lyophilization shelves at −50° C.±3° C. Allow the resuspension to freeze until all product probes reach ≦−40° C.

Lyophilization Cycle Parameters

1. Loading Temperature: −50° C. (product probes <−40° C.)

2. Freezing Hold Time: 30 min

3. Chill condensers to <−55° C.

4. Evacuate system to 75 mTorr and maintain with N₂ bleed.

5. Shelf Temperature Setpoint: 40° C.

6. Shelf Temperature Ramp Rate: 10° C./hour

7. Hold Shelf Temperature at 40° C. for 26 hours.

8. Shelf Temperature Setpoint: 20° C.

9. Shelf Temperature Ramp Rate: 20° C./hour

10. Hold Shelf Temperature at 20° C. for 1 hour.

Lyophilization primary drying is performed at 40° C. for a minimum of 26 hours in a small lyophilizer. Secondary drying is performed at 20° C. to equilibrate the foams at room temperature, as the product will have all free water removed during primary drying.

3. Dry Heat Treatment

a. The lyophilization plugs require drying at a temperature of 100±5° C. for a minimum of 7 hours.

4. Cooling and Equilibration

Upon completion of the dry heat treatment, the foam plugs are cooled under 40% relative humidity for a minimum of 4 hours because if they are packaged right after the dry heat treatment then the foam will be dry and brittle.

5. Compression and Packaging

a. The foam plugs are compressed between 2 non-stick surfaces mechanically separated by metallic shims to prevent crushing of the foam plugs. The foams are compressed to a designed gap of about 0.25 cm thickness. The compression allows for a designed re-expansion of a final thickness of approximately 0.3 cm.

The final product of the above process is a Seprapack™ foam that is significantly more flexible than HA/CMC film to allow for the manipulation and placement of the foam without cracking, even at low humidity. Upon hydration, Seprapack™ swells and will retain about 30 ml water/gm of biomaterial. The process allows for a finished nasal plug that can be:

Easily squeezed, manipulated and cut to size by a physician,

Easily handled and administered in either a wet or dry condition,

Easily contours to a wide range of nasal cavities (up to 1 cm),

Re-expands to greater than 90% of its original pre-compressed height of 1 cm,

Completely bioresorbable, nullifying a second patient visit,

Prevents adhesions,

Minimizes swelling and edema, and controls minimal bleeding

3. In Vitro Testing

In vitro safety testing was conducted for Seprapack™ under ISO 10993 and FDA G95-1 guidelines. Seprapack™ had low endotoxin levels, and did not elicit a cytotoxic response. Likewise, Seprapack™ did not show an in vitro increase in Staphylococcus aureus growth or toxin production. This in vitro testing of Seprapack™ indicates that it is safe for a nasal dressing/sinus stent indication.

4. In Vivo Testing

Anesthetized New Zealand White rabbits received a bilateral wounding of the mucosal tissue surrounding the sinus ostia. One side was left untreated or was treated with Gelfoam® absorbable gelatin (Pharmacia Corporation), a commonly used sinus packing material. The contralateral side was treated with Seprapack™. Gross observations, three days post surgery, revealed presence of test material and no differences in healing among the treatment groups. Microscopically the inflammatory response in the injured mucosa was typical for the acute phase of wound healing. A mild mixed cellular infiltrate that was predominantly neutrophils was present. This was essentially the same for all groups and did not change in the presence of Seprapack™. There was no evidence of giant cells or encapsulation of the packing material, two key hallmarks of a foreign body reaction. Seprapack™ was therefore shown to be safe in short-term in vivo testing.

Generally speaking with regard to surgery anywhere in the body, the time period required to effectively prevent adhesion between tissues will vary according to the type of surgery, the type of tissues involved or injury involved. Generally, the tissues should remain separated for at least 48 hours, and preferably, for a period of at least 7 days. Accordingly, the rate of bioabsorption of the composition used in any particular situation can be varied, for example, by altering the extent of the composition's solubility or insolubility, by varying the density of the polyanionic polysaccharide used, or by varying the thickness and/or shape of the foam used. These characteristics can be altered by routine procedures, and the properties desired for any type of surgery or trauma for which these compositions are indicated can be determined by routine experimentation using the guidance of the examples described herein. These foam compositions have been found to be especially useful in preventing adhesion between tissues following nasal and sinus surgery. These foam compositions should also be applicable to eye, ear, and throat surgery as well. Depending on the particular surgery for which the foam composition is to be used, the foam composition may be in any desired size and shape suitable to optimize its use, especially for preventing adhesion.

Foams of the present invention can further be used for drug delivery. For example, foam compositions containing water-insoluble polyanionic polysaccharides are useful for sustained release drug delivery. The drug to be delivered can be dispersed within the composition, or can be covalently bonded to the foam as described, for example, in R. V. Sparer et al., 1983, Chapter 6, pages 107-119, in T. J. Roseman et al., Controlled Release Delivery Systems, Marcel Dekker, Inc., New York; and the foam can then be implanted or injected at the locus where delivery is desired.

The invention is not limited by the embodiments described above which are presented as examples only but can be modified in various ways within the scope of protection defined by the appended patent claims.

Thus, while there have been shown and described fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps, which perform substantially the same function in substantially the same way to achieve the same results, are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto. All references cited herein are incorporated in their entireties by reference.

We I claim:

1. A method of making a flexible foam, bioresorbable composition comprising the steps of:
a) obtaining a suspension in water of about 1.5 to about 3.0 dry weight % of a reaction product of a polyanionic polysaccharide and a reactant selected from the group consisting of hyaluronic acid, a derivative of hyaluronic acid, and a salt thereof;
b) freezing the suspension at or below 0° C.;
c) lyophilizing the frozen suspension to form a lyophilized product;
d) heating the lyophilized product at about 100° C. for at least about 7 hours; and
e) exposing the heat treated, lyophilized product to air at a relative humidity of about 40% for at least about 4 hours.

2. The method of claim 1, wherein the polyanionic polysaccharide is selected from the group consisting of carboxymethylcellulose, carboxymethylamylose, chondroitin-6-sulfate, chondroitin-4-sulfate, dermatin sulfate, alginate, heparin, and heparin sulfate.

3. The method of claim 1, wherein the polyanionic polysaccharide is carboxymethylcellulose.

4. The method of claim 1, wherein the polyanionic polysaccharide is activated by an activating agent.

5. The method of claim 4, wherein the activating agent is a carbodiimide.

6. The method of claim 3, wherein the reaction product contains about 22 volume % to about 45 volume % of carboxymethylcellulose.

7. The method of claim 6, wherein the reaction product contains about 49 volume % to about 73 volume % of sodium hyaluronate.

8. The method of claim 1, wherein the reaction product is a reaction product of sodium hyaluronate, carboxymethylcellulose and 1-ethyl-3-(3-dimethylaminopropyl) carbodiimide hydrochloride.

9. A method of claim 8, wherein step a) is obtaining a suspension in water of about 2.0 dry wt % of a reaction product of sodium hyaluronate, carboxymethylcellulose and 1-ethyl-3-(3-dimethylaminopropyl) carbodiimide hydrochloride.

10. A method of claim 1, wherein step b) is performed at about −40° C.

11. A flexible foam, bioresorbable composition made by the process of claim 1.

12. A flexible foam, bioresorbable composition made by the process of claim 8.

13. A method of preventing adhesion following surgery, comprising inserting between two tissue surfaces of a patient a foam packing device comprising a flexible foam, bioresorbable composition of claim 11.

14. The method of claim 13, wherein the method comprises inserting the foam packing device into the nasal septum.

15. The method of claim 13, wherein the method comprises inserting the foam packing device into the eye, ear or throat.

16. A method of preventing adhesion following surgery, comprising inserting between two tissue surfaces of a patient a foam packing device comprising a flexible foam, bioresorbable composition of claim 12.

17. The method of claim 16, wherein the method comprises inserting the foam packing device into the nasal septum.

18. The method of claim 16, wherein the method comprises inserting the foam packing device into the eye, ear or throat.

19. A device for implantation within the body for continuous delivery of a drug into the body, comprising a drug and a flexible foam, bioresorbable composition made by the method of claim 1.

20. A method for the release of a drug comprising administering to a mammal in need of treatment with the drug a flexible foam, bioresorbable composition containing the drug, wherein the composition is made by the method of claim 1.

21. A device for implantation within the body for continuous delivery of a drug into the body, comprising a drug and a flexible foam, bioresorbable composition made by the method of claim 9.

22. A method for the release of a drug comprising administering to a mammal in need of treatment with the drug a flexible foam, bioresorbable composition containing the drug, wherein the composition is made by the method of claim 9.
